(19) United States
(12) Patent Application Publication
(10) Pub. No.: US 2021/0250526 A1
(43) Pub. Date: Aug. 12, 2021

(54) DEVICE FOR CAPTURING A HYPERSPECTRAL IMAGE

(71) Applicant: CARBON BEE, Châteauneuf-sur Isère (FR)

(72) Inventor: Gérald GERMAIN, Châteauneuf-sur Isère (FR)

(21) Appl. No.: 16/645,142

(22) PCT Filed: Sep. 11, 2018
(86) PCT No.: PCT/FR2018/052215

§ 371 Date: Mar. 6, 2020

(30) Foreign Application Priority Data
Sep. 12, 2017 (FR) 1758396

(51) Int. Cl. H04N 5/33; G06K 9/46

(52) U.S. Cl. CPC H04N 5/332; G06K 2009/4657; G06K 9/4661

(57) ABSTRACT

The invention relates to a device for capturing a hyperspectral image, comprising: means for acquiring a diffracted image of a focal plane; means for acquiring at least two non-diffracted images of the focal plane, obtained with different chromatography filters; and means for constructing a hyperspectral image from the different diffractions, comprising a neural network configured to calculate the intensity of each voxel of the hyperspectral image according to: the light intensity in each of the non-diffracted images at coordinates x and y, the weight of each intensity depending on the closeness of the desired wavelength to the colour of the chromatography filter of said non-diffracted image; and light intensities in each of the diffractions of the diffracted image in which the x, y coordinates are dependent on coordinates x, y and λ of the voxel.

### TECHNICAL FIELD

The present invention relates to a device for capturing a hyperspectral image.

The invention finds a particularly advantageous application for on-board systems intended to acquire one or more hyperspectral images.

The invention can be applied to all technical fields using hyperspectral images. Thus, and in a non-exhaustive manner, the invention can be used in the medical field, to carry out phenotyping; in the plant sector for the detection of symptoms of stress, disease or the differentiation of species and in the field of chemical analysis, for concentration measurements.

### PRIOR ART

Within the meaning of the invention, a hyperspectral image comprises three dimensions: two “spatial” dimensions and a third “spectral” dimension expressing the variations in the light reflectance for different wavelengths.

A hyperspectral image is usually coded as voxels. Within the meaning of the invention, a voxel corresponds to a pixel of a focal plane of a scene observed for a particular wavelength. A voxel therefore has three coordinates: the abscissa x and the ordinate y (hereinafter called “spatial coordinates”) illustrating the position of the pixel on the focal plane and a wavelength λ (hereinafter called “spectral coordinate”).

Obtaining this hyperspectral image can be achieved simply by using a succession of separate chromatographic filters and by capturing the image of the scene observed under the respective influence of each of these chromatographic filters. This solution is unsatisfactory because it requires as many chromatographic filters as there are wavelengths to analyze. It follows a significant complexification of the acquisition optics to implement the displacement of the different filters in front of a fixed sensor.

To solve this problem, the doctoral thesis “Non-scanning imaging spectrometry”, Descour, Michael Robert, 1994, The university of Arizona, proposes to acquire a single image of the observed scene containing all the information on the influence of different wavelengths.

This method, called CTIS (for “Computed-Tomography Imaging Spectrometer”), proposes to capture a diffracted image of the focal plane of the observed scene by means of a diffraction grating arranged upstream of a digital sensor. This diffracted image, acquired by the digital sensor, takes the form of multiple projections of the focal plane of the observed scene and contains all of the spectral information.

This CTIS method also makes it possible to instantly acquire, that is to say in a single shot, an image containing all the information necessary to find the hyperspectral image. However, the digital sensor simultaneously acquires the original image and its diffractions, significantly reducing the number of pixels available for each of these elements. The spatial precision thus obtained is relatively low with regard to the requirements of certain applications of hyperspectral imaging.

In addition, although the acquisition is fast, this CTIS method is particularly complex to use because of the process of estimating the hyperspectral image implemented from diffractions. Indeed, the transfer function of the diffraction optic must be reversed to reconstruct the hyperspectral image. Unfortunately, the matrix of this transfer function is only partially defined and the result can only be approached iteratively by inversion methods costly in computation time. This CTIS method has been the subject of numerous research works aimed at improving its implementation. Recently, the scientific publication “Practical Spectral Photography”, published in Eurographics, volume 31 (2012) number 2, proposed an optimized implantation strategy in which the time to obtain a hyperspectral image is 11 min over a powerful computer with 16 processor cores.

As it stands, implementations of the CTIS method do not allow precise hyperspectral images to be obtained quickly (from a spatial or spectral point of view). To overcome processing times, the classic analysis process consists of acquiring the data in situ for processing later. This approach poses many constraints regarding the acquisition procedures and the prior evaluation of the quality of future hyperspectral images. The technical problem of the invention consists in improving the process of obtaining a hyperspectral image by diffraction of the focal plane.

### PRESENTATION OF THE INVENTION

The present invention proposes to respond to this technical problem by using the intrinsic non-linearity of a neural network to obtain the hyperspectral image resulting from the diffracted image.

The use of the neural network makes it possible to multiply the inputs of the neural network without exponentially increasing the complexity of the treatments carried out. Thus, the invention proposes to couple the diffracted image with spatially precise images, obtained using separate chromatographic filters. This “data fusion” improves the spatial accuracy of the hyperspectral image. To this end, the invention relates to a device for capturing a hyperspectral image, said device comprising:
means for acquiring a diffracted image of a focal plane along two or three axes of diffraction; each diffraction of said focal plane making it possible to represent said focal plane from a specific angle; and
means of constructing a hyperspectral image from the different diffractions.

The invention is characterized in that the device comprises means for acquiring at least two non-diffracted images of said focal plane obtained with separate chromatographic filters. Said construction means integrate a neural network configured to calculate an intensity of each voxel of said hyperspectral image as a function of:
a light intensity in each of the non-diffracted images at the x and y coordinates, the weight of each intensity depending on the proximity between the desired wavelength and the color of the chromatographic filter of said non-diffracted image; and
light intensities in each of the diffractions of said diffracted image whose coordinates x, y are dependent on the coordinates x, y and λ of said voxel.

The invention thus makes it possible to correlate the information contained in the different diffractions of the diffracted image with information contained in non-diffracted images.

The diffracted image containing a spatially limited information, but very spectrally complete and the non-diffracted images containing a spatially very complete information, but spectrally limited, the invention makes it possible to extract the best of these two types of information in order to obtain a very precise hyperspectral image spatially and spectrally.

The neural network makes it easy to correlate information by associating it simultaneously according to their influences, that is to say their respective weights with respect to the voxel sought in the hyperspectral image. Within the meaning of the invention, a voxel corresponds to a pixel of the focal plane of the scene observed for a particular wavelength. A voxel therefore has three coordinates: the abscissa x and the ordinate y (hereinafter called “spatial coordinates”) illustrating the position of the pixel on the focal plane and a wavelength λ (hereinafter called “spectral coordinate”).

For each voxel, the relevant pixels of the non-diffracted images are sought as a function of its coordinates. Spatial coordinates can be used directly while the spectral coordinate is used to weigh the interest of the pixels. This weighting is performed as a function of the distance between the desired wavelength and that of the used chromatographic filter.

With regard to the diffracted image, the invention has several methods for determining the relevant pixels. Among these, a learning of the neural network makes it possible to indicate the position of each voxel in one or more diffractions. For example, learning by back propagation of the gradient or its derivatives from calibration data can be used.

The invention thus makes it possible to obtain each voxel of the hyperspectral image more quickly than the iterative matrix resolution methods present in the state of the art. In addition, the determination of each voxel can be carried out independently. As there is no interdependence between the estimates, the invention can thus carry out all the calculations in parallel. This facilitates the installation of the device for capturing the hyperspectral image in an on-board system.

In practice, unlike the conventional state of the art of the CTIS method, the invention makes it possible to obtain a hyperspectral image in real time between two acquisitions of the focal plane of the observed scene. In doing so, it is no longer necessary to postpone the processing of diffracted images and it is no longer necessary to store these scattered images after obtaining the hyperspectral image.

According to one embodiment, the intensity of each voxel is sought in eight chromatic representations according to the following relation:

$$\begin{matrix}{x}_{n} \\ {y}_{n}\end{matrix} = \{ \begin{matrix}x + {x}_{{offset}_{n}} + λ . {λ}_{sliceX} \\ y + {y}_{{offsetY}_{n}} + λ . {λ}_{sliceY}\end{matrix} \}$$

with:
n between 0 and 7;
λ_{sliceX} corresponding to the spectral step constant of the pixel in X of said diffracted image;
λ_{sliceY} corresponding to the constant of the spectral pitch of the pixel in Y of said diffracted image;
x_{0ffsetxn} corresponding to the offset along the X axis of the diffraction n;
y_{0ffsetYn} corresponding to the offset along the Y axis of the diffraction n.

These relationships make it possible to quickly find the intensity of the pixels of interest in each diffraction. Indeed, some pixels can be neglected if the wavelength of the diffracted image is not significant.

According to one embodiment, said intensity of the pixel in each of the diffractions of the diffracted image is sought by producing a convolution product between the intensity of the pixel of said diffracted image and the intensity of its close neighbors in said diffractions of the diffracted image.

This embodiment makes it possible to limit the impact of the precision of the detection of the positioning of the pixel in the different diffractions.

According to one embodiment, said diffracted image and said non-diffracted images are obtained by a set of semi-transparent mirrors so as to capture said focal plane on several sensors simultaneously. This embodiment makes it possible to instantly capture identical plans.

According to one embodiment, said diffracted image and said non-diffracted images are obtained by several juxtaposed sensors, each sensor integrating a preprocessing step aimed at extracting a focal plane present on all of the sensors. This embodiment makes it possible to conserve optical power compared to the embodiment with semi-transparent mirrors. According to one embodiment, three non-diffracted images are obtained by an RGB type sensor. This embodiment makes it possible to obtain several non-diffracted images with a single sensor.

According to one embodiment, a non-diffracted image is obtained by an infrared sensor. This embodiment makes it possible to obtain information invisible to the human eye.

According to one embodiment, a non-diffracted image is obtained by a sensor whose wavelength is between 10,000 nanometers and 20,000 nanometers. This embodiment makes it possible to obtain information on the temperature of the scene observed.

According to one embodiment, a non-diffracted image is obtained by a sensor whose wavelength is between 0.001 nanometer and 10 nanometers. This embodiment makes it possible to obtain information on the X-rays present on the observed scene.

According to one embodiment, said diffracted image is obtained by a sensor comprising:
a first converging lens configured to focus the information of a scene on an aperture;
a collimator configured to capture the rays passing through said opening and to transmit these rays over a diffraction grating; and
a second converging lens configured to focus the rays from the diffraction grating on a collection surface.

This embodiment is particularly simple to perform and can be adapted to an existing sensor.

### SUMMARY DESCRIPTION OF THE FIGURES

The manner of carrying out the invention as well as the advantages which result therefrom will clearly emerge from the embodiment which follows, given by way of indication, but not limitation, in support of the appended figures in which FIGS. 1 to 4 represent:

FIG. 1: a schematic front view of a device for capturing a hyperspectral image according to an embodiment of the invention;

FIG. 2: a schematic structural representation of the elements of the device of FIG. 1;

FIG. 3: a schematic representation of the influence weights of the neural network of FIG. 2; and

FIG. 4: a schematic representation of the architecture of the neural network of FIG. 2.

### WAY TO DESCRIBE THE INVENTION

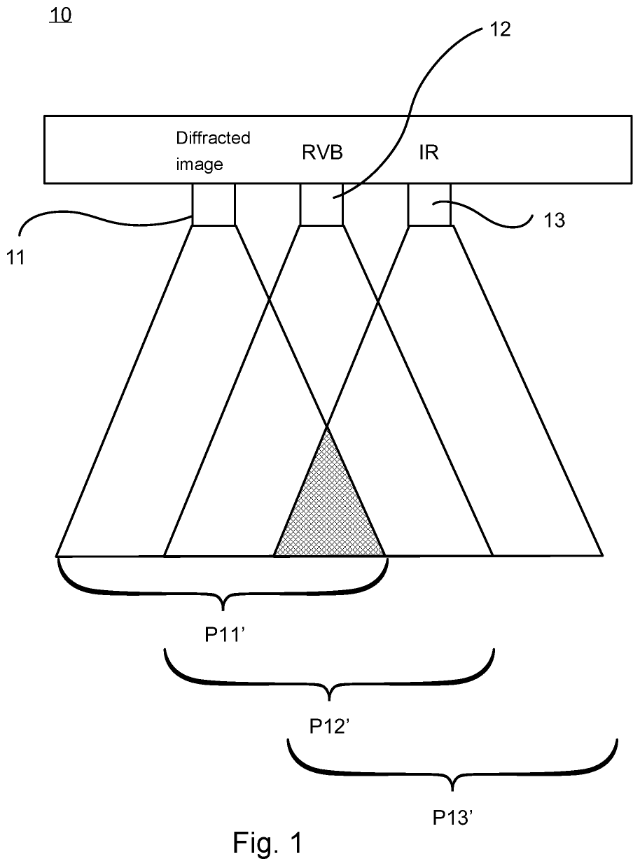

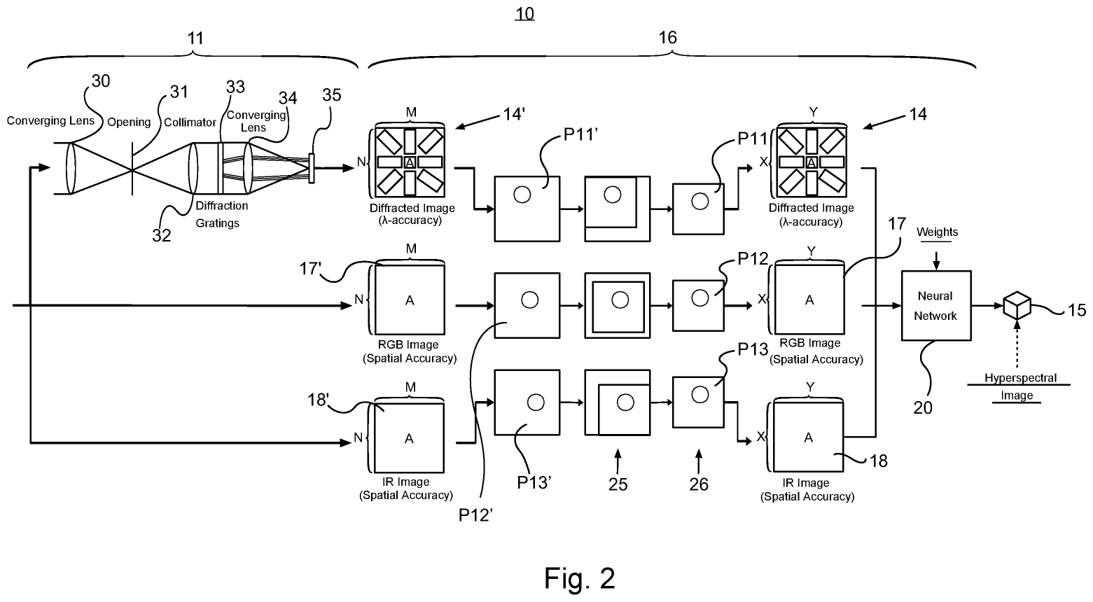

FIG. 1 illustrates a device 10 for capturing a hyperspectral image 15 comprising three juxtaposed sensors 11-13. A first sensor 11 makes it possible to obtain a diffracted image 14′ of a focal plane P11′ of an observed scene. As illustrated in FIG. 2, this first sensor 11 includes a first converging lens 30 which focuses the focal plane P11′ on an opening 31. A collimator 32 captures the rays passing through the opening 31 and transmits these rays to a diffraction grating 33. A second converging lens 34 focuses these rays from the diffraction grating 33 on a collection surface 35.

The structure of this optical network is relatively similar to that described in the scientific publication “Computed-tomography imaging spectrometer: experimental calibration and reconstruction results”, published in APPLIED OPTICS, volume 34 (1995) number 22.

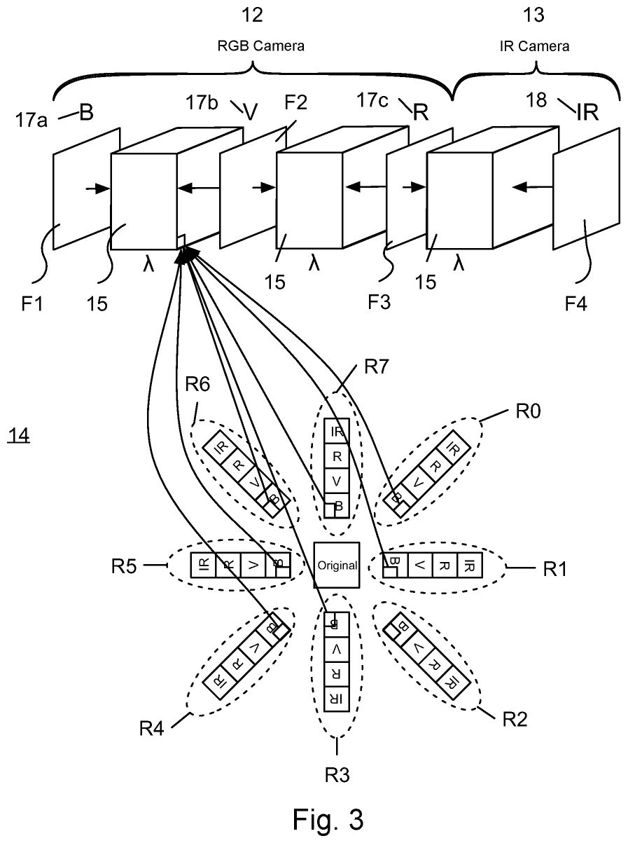

This optical structure makes it possible to obtain a diffracted image 14, illustrated in FIG. 3, having several diffractions R0-R7 of the focal plane P11′ arranged around a non-diffracted image of small size. In the example of FIGS. 1 to 4, the diffracted image presents eight distinct R0-R7 diffractions obtained with two diffraction axes of the diffraction grating 33.

Alternatively, three axes of diffraction can be used on the diffraction grating 33 so as to obtain a diffracted image 14 with sixteen diffractions.

The capture surface 35 can correspond to a CCD sensor (for “charge-coupled device” in English literature, that is to say a charge transfer device), to a CMOS sensor (for “complementary metal-oxide-semiconductor” in the Anglo-Saxon literature, a technology for manufacturing electronic components), or any other known sensor. For example, the scientific publication “Practical Spectral Photography”, published in Eurographics, volume 31 (2012) number 2, proposes to associate this optical structure with a standard digital camera for capturing the diffracted image.

Preferably, each pixel of the diffracted image 14 is coded on 8 bits, thus making it possible to represent 256 colors.

A second sensor 12 makes it possible to obtain a non-diffracted image 17′ of a focal plane P12′ of the same scene observed, but with an offset induced by the offset between the first 11 and the second sensor 12. This second sensor 12 corresponds to an RGB sensor, that is to say a sensor making it possible to code the influence of the three colors Red, Green and Blue of the focal plane P12′. It makes it possible to account for the influence of the use of a blue filter F1, a green filter F2 and a red filter F3 on the observed scene.

This sensor 12 can be produced by a CMOS or CCD sensor associated with a Bayer filter. Alternatively, any other sensor can be used to acquire this RGB image 17′. Preferably, each color of each pixel the RGB image 17′ is coded on 8 bits. Thus, each pixel of the RGB image 17′ is coded on 3 times 8 bits.

A third sensor 13 makes it possible to obtain an infrared image IR 18′ of a third focal plane P13′ of the same scene observed with also an offset with the first 11 and the second sensor 12. This sensor 13 makes it possible to account for the influence of the use of an infrared filter F4 on the observed scene.

Any type of known sensor can be used to acquire this IR 18 image. Preferably, each pixel of the IR image 18 is coded on 8 bits.

The distance between the three sensors 11-13 can be less than 1 cm so as to obtain significant overlap of the focal planes P11′-P13′ by the three sensors 11-13. The topology and the number of sensors can vary without changing the invention.

For example, the sensors 11-13 can acquire an image of the same scene observed by using semi-transparent mirrors to transmit the information of the scene observed to the various sensors 11-13. FIG. 1 illustrates a device 10 comprising three sensors 11-13. As a variant, other sensors can be mounted on the device 10 to increase the information contained in the hyperspectral image. For example, the device 10 can integrate a sensor whose wavelength is between 0.001 nanometer and 10 nanometers or a sensor whose wavelength is between 10,000 nanometer and 20,000 nanometers.

As illustrated in FIG. 2, the device 10 also includes means 16 for constructing a hyperspectral image 15 from the different diffractions R0-R7 of the diffracted image 14 and of the non-diffracted images 17, 18.

In the example of FIGS. 1 to 4, in which the sensors 11-13 are juxtaposed, a preprocessing step is carried out to extract a focal plane P11-P13 present on each of the images 14′, 17′-18′ acquired by the three sensors 11-13. This pretreatment consists, for each focal plane P11′-P13′, of isolating the common part from the focal planes P11′-P13′ then extracting 26 this common part to form the image 14, 17-18 of each focal plane P11-P13 observed by the specific sensor 11-13. The part of each image 14′, 17′-18′ to be isolated 25 can be defined directly in a memory of the sensor device 10 as a function of the positioning choices of the sensors 11-13 between them, or a learning step can be used to identify the part to be isolated 25.

Preferably, the images 17′-18′ from the RGB and IR sensors are cross-checked using a two-dimensional cross-correlation. The extraction of the focal plane of the diffracted image 14′ is calculated by interpolating the offsets in x and y between the sensors 12-13 with respect to the position of the sensor 11 of the diffracted image by knowing the distance between each sensor 11-13. This preprocessing step is not always necessary, in particular, when the sensors 11-13 are configured to capture the same focal plane, for example with the use of semi-transparent mirrors.

When the images 14, 17 and 18 of each focal plane P11-P13 observed by each sensor 11-13 are obtained, the construction means 16 use a neural network 20 to form a hyperspectral image 15 from the information from these three images 14, 17-18′. This neural network 20 aims to determine the intensity I_{χ,γ,λ} of each voxel V_{x,y,λ} of the hyperspectral image 15.

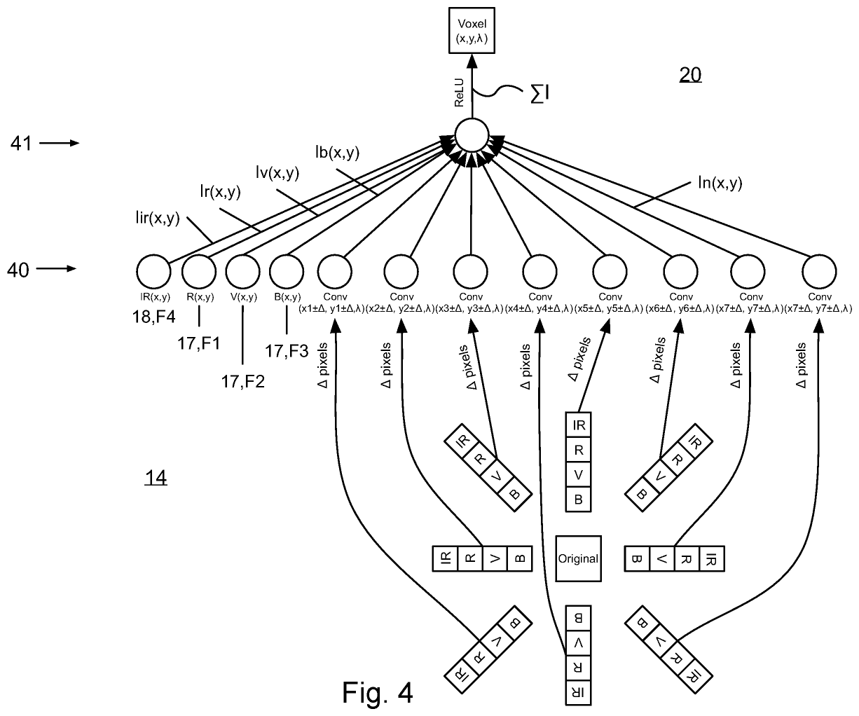

To do this, as illustrated in FIG. 4, the neural network 20 comprises an input layer 40, able to extract the information from the images 14, 17-18, and an output layer 41, able to process these information so as to create information for the considered voxel V_{x,y,λ}.

The first neuron of the input layer 40 makes it possible to extract the intensity I_{IR(x,y)} from the IR image 18 as a function of the x and y coordinates of the sought voxel V_{x,y,λ}. For example, if the IR image 18 is coded on 8 bits, this first neuron transmits to the output layer 41 the 8-bit value of the pixel of the IR image 18 at the sought x and y coordinates.

The second neuron of the input layer 40 performs the same task for the red color of the RGB image 17.

According to the previous example, each color being coded on 8 bits, the sought intensity l_{R(x,y)} is also coded on 8 bits. The third neuron searches for the intensity I_{v(x,y)} in the same way and the fourth neuron searches for the intensity I_{B(x,y)}. Thus, for these first four neurons, it is very easy to obtain the intensity, since it suffices to use the position in x and y of the sought voxel.

The following neurons of the input layer 40 are more complex, since each of the following neurons is associated with an R0-R7 diffraction of the diffracted image 14. These neurons seek the intensity of a specific diffraction l_{n(x,y)} as a function of the position in x and y, but also of the wavelength λ of the voxel sought V_{x,y,λ}.

This relation between the three coordinates of the voxel V_{x,y,λ} and the position in x and y can be coded in a memory during the integration of the neural network 20.

Preferably, a learning phase makes it possible to define this relationship using a known model whose parameters are sought from representations of known objects. An example model is defined by the following relation:

$$\begin{matrix}{x}_{n} \\ {y}_{n}\end{matrix} = \{ \begin{matrix}x + {x}_{{offset}_{n}} + λ . {λ}_{sliceX} \\ y + {y}_{{offsetY}_{n}} + λ . {λ}_{sliceY}\end{matrix} \}$$

with:
n between 0 and 7;
λ_{slicex} corresponding to the constant of the spectral pitch of the pixel along X of said diffracted image;
λ_{sliceY} corresponding to the constant of the spectral pitch of the pixel along Y of said diffracted image;
x_{0ffsetxn} corresponding to the offset along the X axis of the diffraction n;
y_{0ffsetYn} corresponding to the shift along the Y axis of the diffraction n.

A learning phase therefore makes it possible to define the parameters λ_{slicex}, λ_{sliceY}, x_{0ffsetxm}, y_{0ffsetYn} so that each neuron can quickly find the intensity of the corresponding pixel. As a variant, other models are possible, in particular depending on the nature of the used diffraction grating 33.

In addition, the information linked to the intensity of the sought pixel l_{n(x,y)} by each neuron can be determined by a convolution product between the intensity of the pixel of the diffracted image 14 and of its close neighbors in the different R0-R7 diffractions. According to the previous example, the output of these neurons from the input layer 40 is also coded on 8 bits.

All these different intensities of the input layer 40 are injected into a single neuron of the output layer 41 which has the function of sorting out the relevance of all this information and of providing the value of the intensity I_{χ,γ,λ} of the sought voxel V_{χ,γ,λ}. To do this, this output neuron 41 associates a weight with each item of information as a function of the wavelength λ of the sought voxel V_{χ,γ,λ}. Following this modulation on the influence of the contributions of each image 17-18 and of each diffraction R0-R7, this output neuron 41 can sum the contributions to determine an average intensity which will form the intensity I_{χ,γ,λ} of the searched voxel V_{χ,γ,λ}, for example coded on 8 bits. This process is repeated for all the coordinates of the voxel V_{χ,γ,λ} so as to obtain a hypercube containing all the spatial and spectral information originating from non-diffracted images 17-18 and from each diffraction R0-R7. For example, as illustrated in FIG. 3, to find the intensity I_{χ,γ,λ} of a voxel V_{χ,γ,λ} whose wavelength is 500 nm, that is to say a wavelength between blue (480 nm) and green (525 nm).

The output neuron 41 will use the spatial information of the non-scattered images obtained with blue filters F1 and green F2 as well as the information of the different diffractions R0-R7 obtained as a function of the wavelength considered. It is possible to configure the neural network 20 so as not to take certain diffractions R0-R7 into account so as to limit the time for calculating the sum of the contributions. In the example of FIG. 3, the third diffraction R2 is not considered by the neuron of the output layer 41. The weight of each contribution as a function of the wavelength λ of the sought voxel V_{χ,γ,λ} can also be defined during the implantation of the neural network 20 or determined by a learning phase. Learning can be carried out by using known scenes captured by the three sensors 11-13 and by determining the weights of each contribution for each wavelength λ so that the information from each known scene corresponds to the information contained in known scenes.

This learning can be carried out independently or simultaneously with learning the relationships between the three coordinates of the voxel V_{χ,γ,λ} and the position in x and y on the diffracted image 14. This neural network 20 can be implemented in an on-board system so as to process in real time the images from the sensors 11-13 to define and store a hyperspectral image 15 between two acquisitions of the sensors 11-13. For example, the on-board system may include a power supply for the sensors 11-13, a processor configured to perform the calculations of the neurons of the input layer 40 and of the output layer 41 and a memory integrating the weights of each neuron of the input layer 40 as a function of the wavelength λ. As a variant, the different treatments can be carried out independently on several electronic circuits without changing the invention. For example, an acquisition circuit can acquire and transmit the information originating from the neurons of the first layer 40 to a second circuit which contains the neuron of the second layer 41.

The invention thus makes it possible to obtain a hyperspectral image 15 quickly and with great discretization in the spectral dimension. The use of a neural network 20 makes it possible to limit the complexity of the operations to be carried out during the analysis of the diffracted image 14. In addition, the neural network 20 also allows the association of the information of this diffracted image 14 with that of non-diffracted images 17-18 to improve the precision in the spatial dimension.

1. Device for capturing a hyperspectral image, wherein said device comprises:
an acquisition system for acquiring a diffracted image of a focal plane along a number of diffraction axes chosen among the list {two; three}; each diffraction of said focal plane making it possible to represent said focal plane from a specific angle; and
a construction system for constructing a hyperspectral image from the diffractions;
an acquisition system for acquiring at least one non-diffracted images of said focal plane obtained with at least one chronographic filter;
wherein said construction system integrates a neural network configured to calculate an intensity of each voxel of said hyperspectral image as a function:
of a light intensity in each of the non-diffracted images at the coordinates x and y, a weight of each intensity depending on a proximity between a desired wavelength and a color of the chromatographic filter of said non-diffracted image; and
of light intensities in each of the diffractions of said diffracted image whose coordinates u,v are dependent on the coordinates x, y, λ of said voxel.

2. Device according to claim 1, in which the intensity of each voxel is sought in eight chromatic representations according to the following relation:
$$\begin{matrix}{x}_{n} \\ {y}_{n}\end{matrix} = \{ \begin{matrix}x + {x}_{{offset}_{n}} + λ . {λ}_{sliceX} \\ y + {y}_{{offsetY}_{n}} + λ . {λ}_{sliceY}\end{matrix} \}$$
with:
n between 0 and 7;
λ_{sliceX} corresponding to a constant of a spectral pitch of a pixel along X of said diffracted image;
λ_{sliceY} corresponding to a spectral pitch constant of a pixel along Y of said diffracted image;
λ_{0ffsetxn} corresponding to an offset along an X axis of the diffraction n;
y_{0ffsetYn} corresponding to an offset along an Y axis of the diffraction n.

3. Device according to claim 1, in which said intensity of the pixel in each of the diffractions of the diffracted image is sought by producing a convolution product between the intensity of the pixel of said diffracted image and the intensity of its close neighbors in said diffractions of the diffracted image.

4. Device according to claim 1, in which said diffracted image and said non-diffracted images are obtained by a set of semi-transparent mirrors so as to capture said focal plane on several sensors simultaneously.

5. Device according to claim 1, in which said diffracted image and said non-diffracted images are obtained by several juxtaposed sensors, each sensor integrating a preprocessing step aimed at extracting a focal plane present on all the sensors.

6. Device according to claim 1, in which three non-diffracted images are obtained by a sensor of RGB type.

7. Device according to claim 1, in which a non-diffracted image is obtained by an infrared sensor.

8. Device according to claim 1, in which a non-diffracted image is obtained by a sensor whose wavelength is between 10,000 nanometers and 20,000 nanometers.

9. Device according to claim 1, in which a non-diffracted image is obtained by a sensor whose wavelength is between 0.001 nanometer and 10 nanometers.

10. Device according to claim 1, in which said diffracted image is obtained by a sensor comprising:
a first converging lens configured to focus information of a scene on an aperture;
a collimator configured to capture rays passing through said opening and to transmit said rays over a diffraction grating; and
a second converging lens configured to focus rays from the diffraction grating on a collection surface.

11. Method for capturing a hyperspectral image, wherein said method comprises:
an acquisition system acquires a diffracted image of a focal plane along a number of diffraction axes chosen among the list {two; three}; each diffraction of said focal plane making it possible to represent said focal plane from a specific angle; and
a construction system constructs a hyperspectral image from the diffractions;
an acquisition system acquires at least one non-diffracted images of said focal plane obtained with at least one chromographic filter;
said construction system integrates a neural network which calculates an intensity of each voxel of said hyperspectral image as a function:
of a light intensity in each of the non-diffracted images at the coordinates x and y, a weight of each intensity depending on a proximity between a desired wavelength and a color of the chromatographic filter of said non-diffracted image; and
of light intensities in each of the diffractions of said diffracted image whose coordinates u,v are dependent on the coordinates x, v, λ of said voxel.
